(19) United States
(12) Patent Application Publication
(10) Pub. No.: US 2013/0323753 A1
(43) Pub. Date: Dec. 5, 2013

(54) METHODS AND KITS FOR IDENTIFICATION OF ANTI-EXCITOTOXIC COMPOUNDS

(71) Applicant: RUTGERS, THE STATE UNIVERSITY OF NEW JERSEY, (US)

(72) Inventor: Bonnie L. Firestein-Miller, Hillsborough, NJ (US)

(73) Assignee: RUTGERS, THE STATE UNIVERSITY OF NEW JERSEY, New Brunswick, NJ (US)

(21) Appl. No.: 13/870,592

(22) Filed: Apr. 25, 2013

Related U.S. Application Data
(62) Division of application No. 12/676,863, filed on Mar. 5, 2010, now abandoned.
(60) Provisional application No. 60/967,765, filed on Sep. 6, 2007.

(51) Int. Cl. G01N 33/68

(52) U.S. Cl. CPC G01N 33/6896
USPC 435/7.21

(57) ABSTRACT

The instant invention provides methods and kits for screening for anti-excitotoxic compounds which increase the total amount of EAAT-1 protein or the surface amount of EAAT-1 protein.

### CROSS-REFERENCE TO RELATED APPLICATIONS

The present application is a divisional application of U.S. patent application Ser. No. 12/676,863, filed on Mar. 5, 2010, which is a 35 U.S.C. §371(c) National Phase application of International Application Serial No. PCT/US2008/075446 filed on Sep. 5, 2008, which in turn claims priority benefit under 35 U.S.C. §119(e) to U.S. Provisional Patent Application No. 60/967,765 filed on Sep. 6, 2007, the contents of all of which are hereby incorporated by reference.

### FIELD OF THE INVENTION

This invention generally relates to kits and methods for identification of anti-excitotoxic compounds.

### BACKGROUND OF THE INVENTION

Neurons are extremely vulnerable to injury during pathological conditions, including stroke and spinal cord damage. The process of neuronal death resulting from spinal cord injury (SCI) involves glutamate receptor overstimulation as a result of tissue damage, ischemic cell death, and synaptic and non-synaptic transport of glutamate (Liu et al. 1991; Liu et al. 1999; McAdoo et al. 1999). Impaired mitochondrial function and excess calcium influx follows (Choi 1996; Yu et al. 1998), resulting in neuronal apoptosis and necrosis (McAdoo et al. 1999). N-methyl-D-asparate (NMDA) and alpha-hydroxy-5-methyl-4-isoazole-propionic acid (AMPA) receptors have been shown to play central roles in the cellular damage caused by glutamate.

The roles of astroglia after spinal cord injury have been studied rigorously. Astroglia, especially reactive astrocytes, have been found to form the glia scar, generally considered as a major impediment to axon regeneration (Liuzzi and Lasek 1987; Rudge and Silver 1990). However, more recent studies indicate that astrocytes may protect tissue and preserve function after SCI (Faulkner et al. 2004; Silver and Miller 2004).

The two major types of glutamate transporters, EAAT-1 (Glial Glutamate Transporter, GLAST) and EAAT-2 (Glial Glutamate Transporter-1, GLT-1), are primarily expressed in astroglial cells. These transporters have been shown to protect neurons from glutamate toxicity (Rothstein et al. 1993). Moreover, there are studies demonstrating that EAAT-1 and EAAT-2 are acutely up-regulated after spinal cord injury (Vera-Portocarrero et al. 2002).

Many attempts have been undertaken to identify anti-excitotoxic compounds. For example, antagonists to NMDA and AMPA glutamate receptors have shown promising improvements for histological damage and functional deficits in experimental animal models of SCI (Faden et al. 1990; Faden et al. 1988; Gomez-Pinilla et al. 1989; Liu et al. 1997; Wrathall et al. 1997). Nevertheless, these compounds fail to yield successful clinical results (Doppenberg et al. 1997). Therefore, there is a need in the art to identify other neuroprotective and regenerative agents that fight glutamate toxicity after SCI.

### SUMMARY OF THE INVENTION

The instant invention addresses these and other needs of the prior art by providing, in one aspect, a method of identifying compounds reducing excitotoxicity in neurons, the method comprising: a) providing a library of compounds suspected of reducing excitotoxicity in neurons; b) providing a plurality of samples of glial cells; c) contacting at least one member of the plurality of the samples with at least one member of the library of the compounds suspected of reducing excitotoxicity in neurons; d) determining the amount of EAAT-1 protein in said at least one member of the plurality of the samples of glial cells contacted with the at least one member of the library of the compounds suspected of reducing excitotoxicity in neurons; e) comparing the amount of the EAAT-1 protein from step (d) with the amount of EAAT protein from at least one member of the plurality of samples of glial cells not contacted with the at least one member of the library of the compounds suspected of reducing excitotoxicity in neurons; and f) selecting the members of the library of compounds suspected of reducing excitotoxicity in neurons which increase the amount of the EAAT protein in the respective members of the plurality of samples of glial cells.

In another aspect, the invention provides a method of identifying compounds reducing excitotoxicity in neurons, the method comprising: a) providing a library of compounds suspected of reducing excitotoxicity in neurons; b) providing a plurality of samples of glial cells; c) contacting at least one member of the plurality of the samples with at least one member of the library of the compounds suspected of reducing excitotoxicity in neurons; d) determining the amount of EAAT-1 protein present on a cell surface of said at least one member of the plurality of the samples of glial cells contacted with the at least one member of the library of the compounds suspected of reducing excitotoxicity in neurons; e) comparing the amount of the EAAT-1 protein from step (d) with the amount of EAAT-1 protein present on a cell surface of at least one member of the plurality of samples of glial cells not contacted with the at least one member of the library of the compounds suspected of reducing excitotoxicity in neurons; and f) selecting the members of the library of compounds suspected of reducing excitotoxicity in neurons which increase the amount of the EAAT-1 protein overall or on the respective cell surfaces of the respective members of the plurality of samples of glial cells.

These and other embodiments of the methods of the instant invention may be implemented by using the kits of the instant invention. Accordingly, in yet another aspect, the invention provides a kit for identifying compounds reducing excitotoxicity in neurons, the kit comprising: a) a plurality of samples of glial cells; b) a means for determining the amount of EAAT-1 protein present on a cell surface; and c) a set of instructions. In another embodiment, the kit comprises: a) a plurality of samples of glial cells; b) a means for determining the amount of EAAT-1 protein present in a cell; and c) a set of instructions.

### BRIEF DESCRIPTION OF THE FIGURES

FIG. 1 demonstrates that Glutamate induces loss of spinal cord neurons in a dose-dependent manner in cultures spinal cord neurons. (A) Control cultures and cells treated with 500 μM glutamate were illustrated. (B) Numbers of neurons that survived (MAP-2+) were counted.

FIG. 2 demonstrates that addition of uric acid (“UA”) does not affect survival of spinal cord neurons.

FIG. 3 demonstrates that UA blocks glutamate toxicity to spinal cord neurons. Spinal cord neurons were grown in SCM for 6 days before being treated with glutamate for 1 hour, with or without the presence of UA. (A) Control cultures, cells treated with 500 μM glutamate, and cells treated with 500 μM glutamate and 100 μM UA (added together) are illustrated. (B) When glutamate and UA were added together, UA blocked glutamate toxicity. (C) When UA was added after glutamate treatment, it had similar effects to reverse glutamate damage.

FIG. 4 is a characterization of cell types present in cultures grown in serum-containing medium (spinal cord medium; SCM) and cultures grown in Neurobasal medium (NB) and treated with Ara-C. Cells grown in SCM were mixed cultures which included neurons, mature and immature astrocytes, and oligodendrocytes. Cells grown in NB and treated with Ara-C were a pure neuronal population.

FIG. 5 demonstrates a dose-dependent cell loss in pure spinal cord neuron cultures to glutamate toxicity. Pure spinal cord neuron cultures were established, and after 6 days, these cells were treated with glutamate for 1 hour. Cells were fixed after 24 hours and stained with anti-MAP-2 antibody. (A) Control cultures and cells treated with 2, 10, and 500 μM glutamate are illustrated. (B) Numbers of neurons that survived (MAP-2+) were counted.

FIG. 6 demonstrates that UA does not decrease glutamate toxicity in pure spinal cord neuron cultures.

FIG. 7 demonstrates that UA reduces damages elicited by Sin-1 treatment in pure spinal cord neuron cultures. (A) UA (100 μM) was added with and after glutamate treatments. (B) UA (100 μM) was added only after glutamate treatments.

FIG. 8 demonstrates that conditioned medium (CM) from astroglial cultures does not reduce glutamate toxicity to pure spinal cord neurons. CM was collected from pure spinal cord astroglial cultures grown in NB and treated with UA or Locke's buffer. CM1 was from Locke's buffer treated group. CM2 was from UA treated group (containing UA). Pure neuron cultures were treated with glutamate (10 μM) for 1 hour and medium was changed to CM.

FIG. 9 demonstrates that re-plating of astroglial cells in pure neuron cultures reinstates the effects of UA in reducing glutamate toxicity. Pure astroglial cultures were re-plated in pure spinal cord neuron cultures grown in SCM for 5 days. After 24 hours, cells were exposed to glutamate (50 μM) for 1 hour and UA (100 μM) was added after the medium was changed.

FIG. 10 demonstrates that EAAT-1 is expressed by GFAP+ astroglia, EAAT-2 is expressed by vimentin+ astroglia, and treatment with the EAAT inhibitor THA blocks the effects of UA to protect neurons against glutamate toxicity. (A) Mixed cultures derived from spinal cords of E16 rats were fixed on DIV 7. Cells were double labeled for EAAT-1 and GFAP or EAAT-2 and vimentin. Scale bar, 50 μm. (B) THA (50 μM) was added to DIV 6 mixed cultures one hour prior to a one-hour exposure to glutamate (50 μM). THA (50 μM) and UA (100 μM) were added when the medium was changed.

FIG. 11 demonstrates that protein expression of EAAT-1 is upregulated by treatment of UA. (A), Western blot of EAAT-1, EAAT-2 and actin (internal control). (B, C), Quantitative analysis of EAAT-1 (B) and EAAT-2 (C) expression normalized to actin protein levels.

### DETAILED DESCRIPTION OF THE INVENTION

Generally, this invention is based on a surprising discovery that uric acid mediates its neuroprotective effect, at least in part, through the increase of the amount of EAAT-1 protein. As discussed in the Examples of the instant application, the different levels of damage elicited by glutamate in mixed and pure neuron cultures suggest a beneficial role played by astroglia in the acute phase of SCI. This surprising discovery reveals an intriguing prospect that astroglia mediate the effects of UA to reduce glutamate-elicited damage to neurons, casting new insight into possible roles astroglia can play in the anti-excitotoxic process after trauma. Furthermore, the results disclosed in this application suggest that UA can act as a neuroprotective agent after glutamate exposure, further supporting its use as a therapeutic agent after SCI.

Additionally, previous studies used a pre-treatment and concurrent treatment paradigm, which is an issue for therapeutic use of UA. These studies show that UA can be used as a neuroprotective agent after injury, making it a viable treatment for SCI.

Accordingly, without wishing to be bound by a particular theory, the inventors have concluded that compounds that increase the amount of EAAT-1 protein within a glial cell or on a surface of a glial cell, would have anti-excitotoxic properties. As used in this disclosure, the term “EAAT-1” refers to a human EAAT-1 protein, e.g., having GenBank Accession NO: P43003.1 (SEQ ID NO: 1) or a mammalian homolog thereof. The sequences of EAAT-1 homologs from different species is available from GenBank. For example and without limitation, the term “EAAT-1” also includes Accession No. NP 683740.1 (mouse EAAT-1 homolog, SEQ ID NO: 2), and Accession No. P24942.2, (rat EAAT-1 homolog, SEQ ID NO: 3)

Thus, in one aspect, the instant disclosure is drawn to a method of identifying compounds reducing excitotoxicity in neurons, the method generally comprising providing a library of compounds and contacting the compounds in the library with the glial cells. After the compound or compounds have been introduced to the cells, the amount of EAAT-1 protein in the cell or on the surface of the cell is quantified and compared with the amount of EAAT-1 protein in a control cell or on the surface of the control cell which has not been contacted with any compound in the library.

Generally, a plurality of assays can be run in parallel with different concentrations of the compounds provide din the library to obtain a differential response to the various concentrations. Typically, one of these concentrations serves as a negative control, i.e., at zero concentration or below the level of detection.

Although the screening methods are generally used as an assay to identify previously unknown molecules that can act as a therapeutic agent, the method can also be used to confirm and standardize the desired activity of compounds known to increase the total or surface amount of EAAT-1 protein or to optimize the structure and/or activity of compounds known to increase the total or surface amount of EAAT-1 protein during, e.g., molecular evolution procedures.

In one embodiment, uric acid can also be included within the library of the compounds according to the instant invention. The uric acid would serve as a positive control.

Uric acid has the formula shown below, i.e.,

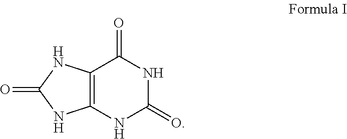 [SMILES: O=c1[nH]c(=O)c2[nH]c(=O)[nH]c2[nH]1]

Since the inventors have surprisingly discovered that uric acid exerts its neuroprotective effect at least in part by increasing the total amount of EAAT-1 protein in glial cells, the library of the compounds may comprise compounds related to uric acid. For example, the library may comprise, or, optionally, consist of, the compounds encompassed by Formula II below:

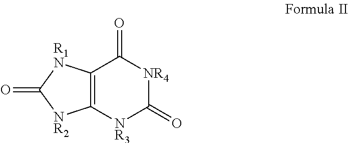

wherein R₁, R₂, R₃, and R₄ are independently selected from hydrogen, halogens, glycosides, substituted and unsubstituted C₁-C₆ alkyls, NO₂, H₂PO₄, SO₂R₅, OR₅, SR₅, COOR₅, COR₅, NR₆R₇, COR₈, N═R₉, and C═R₁₀, wherein R₅, R₆, R₇, R₈, R₉ and R₁₀ are independently selected from hydrogen and substituted and unsubstituted C₁-C₃ alkyls.

Multiple types of glial cells are suitable for the instant invention. Generally, glial cells include astroglial cells (comprising astrocytes), oligodendrocytes, ependymal cells, Schwann cells, satellite cells, and microglial cells. Astrocytes are the most abundant type of macroglial cells, and they express high levels of EAAT-1 compared to other cell types. Accordingly, in a preferred embodiments, the glial cells of the instant invention are astrocytes.

Conveniently, multiple immortalized glial cell lines have been described. For example, the rat glial cell line 5.5B8 has phenotypic characteristics of both oligodendrocytes and astrocytes, with expression of MBP and 2′,3′-cyclic nucleotide 3′-phosphodiesterase (CNP), and low, but detectable, expression of glial fibrillary acidic protein (GFAP) and the lipids or proteins recognized by the mAbs A2B5 and O4 (Bozyczko, et al., Ann. NY Acad. Sci., 605:350-353 (1990)). Additional examples of immortalized astrocytes are disclosed in US Patent Publication 20080200412 and include T98G, G18, U251, H4 cell lines. Further examples have been disclosed in Sacchettoni et al., GLIA 1998; 22(1):86-93, and Wang, J-H et al., Soc. Neurosci 28:1540, 1998. Astrocytes derived from glia-restricted precursors, (GDAs), described, e.g., in Davies et al., J. Biol. 5(3): 7-27 (2006), are also suitable for the instant invention.

The amount of EAAT-1 protein can be detected by many techniques known to those of ordinary skill in the art. The choice of the particular technique will ultimately depend on the size of the library, the amount of the glial cells in the sample, and the decision of the practitioner regarding the measurement of total amount of the EAAT-1 protein as opposed to the measurement of surface the amount of the EAAT-1 protein.

In embodiments entailing the measurement of the total amount of the EAAT-1 protein, one may conveniently use cytoplasmic or whole-cell extracts of the glial cells present in the sample. In embodiments entailing the measurement of the surface amount of the EAAT-1 protein, in situ immunostaining or membrane extracts can be used.

Preferably the measurements of the total or the surface amount of the EAAT-1 protein are performed by an antibody-based method. The antibodies to EAAT-1 are well known and are commercially available, from e.g., Abcam, Inc. (Cambridge, Mass.), Calbiochem Inc. (La Jolla, Calif.) or other commercial suppliers.

Alternatively, antibodies can be made by immunizing a suitable subject, such as a rabbit, with EAAT-1 (preferably mammalian; more preferably human) or an antigenic fragment thereof. The antibody titer in the immunized subject may be monitored over time by standard techniques, such as with ELISA, using immobilized marker protein. If desired, the antibody molecules directed against EAAT-1 may be isolated from the subject or culture media and further purified by well-known techniques, such as protein A chromatography, to obtain an IgG fraction, or by affinity chromatography, as described in Firestein et al., Neuron 24:659 (1999).

As an alternative to preparing monoclonal antibody-secreting hybridomas, a monoclonal antibody to EAAT-1, or a fragment thereof, may be identified and isolated by screening a recombinant combinatorial immunoglobulin library (e.g., an antibody phage display library) to thereby isolate immunoglobulin library members that bind to EAAT-1, or a fragment thereof. Kits for generating and screening phage display libraries are commercially available from, e.g., Dyax Corp. (Cambridge, Mass.) and Maxim Biotech (South San Francisco, Calif.). Additionally, examples of methods and reagents particularly amenable for use in generating and screening antibody display libraries can be found in the literature.

Fragments of antibodies to EAAT-1, may be produced by cleavage of the antibodies in accordance with methods well known in the art. For example, immunologically active F(ab′) and F(ab′)₂ fragments may be generated by treating the antibodies with an enzyme such as pepsin. Additionally, chimeric, humanized, and single-chain antibodies to EAAT-1, comprising both human and nonhuman portions, may be produced using standard recombinant DNA techniques. Humanized antibodies to EAAT-1 may also be produced using transgenic mice that are incapable of expressing endogenous immunoglobulin heavy and light chain genes, but which can express human heavy and light chain genes.

In the immunological assays of the present invention, the EAAT-1 polypeptide is typically detected directly (i.e., the anti-EAAT-1 antibody is labeled) or indirectly (i.e., a secondary antibody that recognizes the anti-EAAT-1 antibody is labeled) using a detectable label. The particular label or detectable group used in the assay is usually not critical, as long as it does not significantly interfere with the specific binding of the antibodies used in the assay.

In one embodiment, the anti-EAAT-1 antibody may be modified with a label and thus may be detected directly. In another embodiment, a secondary antibody, which binds the anti-AAT-1 antibody, is labeled. As is well known to one of skill in the art, a secondary antibody is chosen that is able to specifically bind the specific species and class of the anti-EAAT-1 antibody. For example, if the anti-EAAT-1 antibodies are human IgGs, then the secondary antibody may be an anti-human-IgG. The amount of an antibody-receptor complex in the biological sample can be detected by detecting the presence of the labeled secondary antibody.

Suitable labels are widely known in the art and include various enzymes, prosthetic groups, fluorescent materials, luminescent materials, magnetic agents and radioactive materials. Examples of suitable enzymes include horseradish peroxidase, alkaline phosphatase, p-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, diclilorotriazinylamine fluorescein, dansyl chloride or phycoerythrin; examples of a luminescent material include luminol luciferin, pyrogallol, or isoluminol; an example of a magnetic agent includes gadolinium; and examples of suitable radioactive material include ¹²⁵I, ¹³¹I, ³⁵S or ³H. Other molecules that can bind to antibodies include, without limitation, Protein A and Protein G, both of which are available commercially, for example, from Pierce Chemical Co. (Rockford, Ill.)

Exemplary detection methods suitable for the instant invention include, without limitations, immunoblot, competition or sandwich ELISA, a radioimmunoassay, a dot blot assay, a fluorescence polarization assay, a scintillation proximity assay, a homogeneous time resolved fluorescence assay, a resonant mirror biosensor analysis, immunostaining, and a surface plasmon resonance analysis. Exemplary non-limiting embodiments of these methods are discussed below.

Immunoblot (Western Blot)

In this assay, the cell extract (e.g., the whole cell extract, the membrane extract, or the cytoplasmic extract) is run on a gel and then transferred onto a membrane (e.g., nitrocellulose membrane). The nitrocellulose membrane is then contacted with the anti-EAAT-1 antibody, which, optionally may be labeled. If the EAAT-1 antibody is unlabeled, the resulting complex is then incubated with a labeled secondary antibody. The amount of the label allows to determine the amount of the EAAT-1 protein in the cell extract.

Competition ELISA.

In the competition immunoassay method, antibody bound to a solid surface is contacted with a sample containing an unknown quantity of EAAT-1 and with labeled EAAT-1. The amount of labeled EAAT-1 bound on the solid surface is then determined to provide an indirect measure of the amount of EAAT-1 in the sample.

Sandwich ELISA.

In one embodiment of a Sandwich ELISA, the primary antibody is immobilized on a solid carrier and is brought into contact with the cell extract. After sufficient incubation time, which may be determined by routine experimentation, the quantity of the bound EAAT-1 protein is determined by adding a second antibody which is labeled with a detectable label such as a radioactive atom, a fluorescent or luminescent group or, in particular, an enzyme (for example horseradish peroxidase (HRP)). If the modified antibody candidates are human IgGs, then the second antibody may be an anti-human-IgG antibody.

The amount of the bound second antibody is then determined by measuring the activity, for example, the enzyme activity of the label. This activity is a measure of the amount of the EAAT-1 protein.

Dot Blot Analysis.

A dot blot procedure can also be used for this analysis. The use of the dot blot procedure eliminates the need to perform electrophoresis and allows rapid analysis of a large number of samples. In one embodiment of this method, the aliquots of the cell extracts can be placed on a membrane, such as, for example, nitrocellulose membrane, and contacted with the primary antibody. The resulting complex is then incubated with a radioactively or fluorescence labeled secondary antibody. The amount of signal produced by the label (radioactivity, light, color, etc) can then be quantified.

Fluorescence Polarization Assay.

This assay is based on the principle that a fluorescent tracer, when excited by plane polarized light of a characteristic wavelength, will emit light at another characteristic wavelength (i.e., fluorescence) that retains a degree of the polarization relative to the incident stimulating light that is inversely related to the rate of rotation of the tracer in a given medium. As a consequence of this property, a tracer substance with constrained rotation, such as in a viscous solution phase or when bound to another solution component, such as an antibody with a relatively lower rate of rotation, will retain a relatively greater degree of polarization of emitted light than if in free solution. Thus, a person of skill in the art can label the anti-EAAT-1 antibody with an appropriate label and contact the labeled antibody with the cell extract containing EAAT-1 protein. The fluorescence polarization assays can be conducted in commercially available automated instruments such as IMx®, TDx®, and TDxFLx™. (Abbott Laboratories, Abbott Park, Ill.).

Scintillation Proximity Assay.

The anti-EAAT-1 antibodies can be coupled to a scintillation-filled bead. Binding of radio-labeled EAAT-1 would result in emitted radioactivity which can be quantified on a scintillation counter. Commercial kits for the scintillation proximity assay are currently available and may be purchased from, for example, Amersham Life Science (Piscataway, N.J.).

Homogeneous Specific Binding Assay

In this assay, a conjugate is formed between a binding substance and coupled to a label, which is chosen in such a way that it behaves differently depending on whether the binding substance is bound or free. Thus, in one embodiment of the method, the samples comprising EAAT-1 protein can be impregnated into a solid carrier and contacted with different liquid samples containing known amounts of the anti-EAAT-1 antibodies which are labeled. Examples of labels suitable for this method are chemiluminescent compounds and enzymes, as disclosed above. Change in chemiluminescence can be measured, thus reflecting on the relative amount of bound modified antibody candidates.

Surface Plasmon Resonance Analysis

This method is based on quantifying the intensity of electromagnetic waves, also called surface plasmon waves, which may exist at the boundary between a metal and a dielectric. Such waves can be exited by light which has its electric field polarized parallel to the incident plane (i.e., transverse magnetic (TM) polarized). In this method, one of the reagents (i.e., the samples containing EAAT-1 protein or the anti-EAAT-1 antibody) is coupled to the dextran layer (covering the metal film) of a sensor chip and solutions containing different concentrations of the other reagent (i.e. the anti-EAAT-1 antibody of the cell extract containing EAAT-1 protein or the inhibitory receptor or the antibody, respectively) are allowed to flow across the chip. Binding (association and dissociation) is monitored with mass sensitive detection. BIACORE® (Biacore AB, Uppsala, Sweden) equipment can be used for this method.

Immunostaining

This method differs from the other methods disclosed above since whole cells are used rather than cell extracts. The cells are fixed in situ and contacted with an anti-EAAT-1 antibody, followed by contacting the cells with a secondary antibody which is labeled. The intensity of the signal is quantified, thus providing information about the amount of the EAAT-1 protein present at the cell surface.

Other modifications of these assays, not disclosed in this application will be apparent to a person of ordinary skill in the art. The claims of the present invention include all such modifications.

The methods according to any of the embodiments of the instant invention can conveniently be practiced by using the kits of the instant invention. Generally, the kits comprise a source of glial cells, a means for detection the total amount of the EAAT-1 protein or a surface amount of the EAAT-1 protein, and a set of instruction.

In one set of embodiments, the source of glial cells may be present in a plurality of samples (e.g., 12 plate wells or the like) which can be shipped in an appropriate medium. Alternatively, the cells are supplied in one reservoir (e.g., a flask), and are later split into multiple samples by the user.

The means for detection of the total or the surface amount of the EAAT-1 protein preferably comprise at least the anti-EAAT-1 antibodies, and may also comprise different labels, secondary antibodies, enzyme substrates, solid supports (e.g., beads or membranes), cell culture media, buffers for preparing extracts of different cell fractions, and other components. The kits may also comprise urea, which would serve as a positive control.

The set of instructions would generally provide information for safe and efficient use of the kit. The instructions may be in any medium, including, without limitations, printed, audio-, video- and electronic media.

Specific embodiments according to the methods of the present invention will now be described in the following examples. The examples are illustrative only, and are not intended to limit the remainder of the disclosure in any way.

### EXAMPLES

The following materials and methods are applicable to all of the following examples unless the examples specifically note otherwise.

Spinal Cord Neuron Cultures:

Spinal cords were dissected from embryonic day 16 (E16) rat embryos. Meninges were removed, and the cords were dissociated with gentle trituration. Cells were plated at a density of 350 neurons/mm². The mixed cultures were grown in serum-containing medium (SCM; 89.4% MEM (Minimum Essential Medium; Gibco), 10% horse serum, 0.6% glucose, supplemented with penicillin and streptomycin) for 6 days at 37° C. and 5% CO₂ before treatment.

To grow pure spinal neuron cultures, SCM was changed to Neurobasal medium (NB; Neurobasal medium (Gibco) supplemented with B-27, penicillin, and streptomycin) at 24 hours after plating. After an additional 24 hours, cytosine arabinoside (Ara-C, 5 μM) was added to these cultures for 3 days after which the Ara-C containing media was changed to fresh NB media. Cells were treated 24 hours later.

Spinal cord astroglial cultures were derived from postnatal day 1 rats using a modified approach from MaCarthy and de Vellis (McCarthy and deVellis 1980). Spinal cords were dissected and dissociated with trituration. Cells were plated in tissue culture flasks at density of 1000 cells/mm². Cultures were grown in NM-15 medium (84.4% MEM, 15% Fetal Bovine Serum, 0.6% glucose, supplemented with penicillin and streptomycin) for 9 days. Medium was changed every 3 days. The flasks were then shaken at 400×rpm for 20 minutes before the medium was changed and the floating cells (mostly microglia) were washed away. The flasks were again shaken overnight at 250×rpm to remove attached oligodendrocytes and remaining microglia. Following shaking, floating cells were washed away and medium was changed. Ara-C was added to final concentration of 10 μM to reduce the number of undifferentiated cells. Cultures were grown for 3 additional days before being trypsinized and re-plated at a density of 500 cells/mm² in SCM. These cultures only contain GFAP+ and vimentin+ astroglia. Staining for MAP-2 and CNPase were negative.

To examine whether soluble factors from astroglia are involved in actions of UA, astroglial cultures were grown in SCM for 3 days before the medium was changed to NB. After another 3 days, UA was added to some cultures while other cultures were treated with Locke's buffer as vehicle for 24 hours. Conditioned medium (CM) from all cultures was collected. CM from UA treated group still contains UA, while CM from the control group does not. The different CM was used in experiments to evaluate the possible involvement of UA-elicited soluble factors.

To determine the contribution of direct astroglial cell presence to the neuroprotective effects of UA, astroglial cultures were grown for 5 days in SCM before the cells were trypsinized and plated onto pure spinal cord neuron cultures grown for 5 days in vitro (DIV 5). The co-culture was grown in SCM and treated 24 hours later.

Treatments:

Reagents used to treat cells were made into stock solutions. Glutamate and uric acid (UA) were dissolved in Locke's buffer (NaCl, 154 mM; KCl, 5.6 mM; CaCl2, 2.3 mM; MgCl₂, 1.0 mM; NaHCO3, 3.6 mM; glucose, 5 mM; Hepes, 5 mM; pH 7.2) and diluted as indicated. To induce peroxynitrite toxicity, Sin-1 (3-morpholinoesydnonimine; Sigma), a peroxynitrite donor, was dissolved in PBS and used to treat cells at the indicated concentrations. L-Threohydroxy aspartate (THA; Sigma), an inhibitor of EAATs, was used to block glutamate transporter function. THA was dissolved in PBS.

When cells were treated with glutamate, the medium was replaced with solutions of glutamate in Locke's buffer, with or without UA, for 1 hour at 37° C. In control cultures, the medium was replaced with Locke's buffer. After 1 hour, glutamate solutions were changed to SCM or NB and UA was added at the same concentration. In all cases, same amount of Locke's buffer was added to control as vehicle. When the cultures were treated with Sin-1, the drug was added directly to the medium with or without UA, and the same amount of PBS and Locke's buffer were added to control as vehicle. In all cases, cells were incubated for another 24 hours at 37° C. before being fixed or harvested.

Immunocytochemistry:

Specific primary antibodies were used to identify different type of cells in the culture using immunocytochemistry. Monoclonal anti-MAP-2 (microtubule associated protein 2; BD Pharmingen) was used to identify neurons; monoclonal anti-GFAP (glial fibrillary acidic protein; Chemicon Inc.) was used to identify astrocytes; monoclonal anti-CNPase (2′3′-cyclic-nucleotide 3′-phosphodiesterase; Chemicon Inc.) was used to identify oligodendrocytes; monoclonal anti-vimentin (Chemicon Inc.) was used to identify astrocyte progenitors; and monoclonal anti-CD11 (clone OX42; Serotec Inc.) was used to identify microglia.

Cells were fixed with 4% paraformaldehyde for 30 minutes at room temperature. After washing with PBS, the cultures were incubated with anti-MAP-2 (1:500), anti-GFAP (1:1000), anti-CNPase (1:500), anti-vimentin (1:1000) or anti-CD11 (1:500) for 2 hours at room temperature. Positive cells were then visualized with Cy2 anti-mouse secondary antibodies.

For double labeling of GFAP and EAAT-1 or vimentin and EAAT-2, fixed cells were incubated with mixed solutions of monoclonal anti-GFAP (1:1000) and guinea pig polyclonal anti-EAAT-1 (Chemicon Inc., 1:1000) or mixed solutions of monoclonal anti-vimentin (1:2000) and guinea pig polyclonal anti-EAAT-2 (Chemicon Inc., 1:1000) at room temperature for 2 hours. After rinsing, cultures were visualized with Cy2 anti-mouse and Cy3 anti-guinea pig secondary antibodies.

Western Blot Analysis:

At DIV 7, control and treated mixed spinal cord cultures grown on 35 mm dishes were scraped and protein was harvested in TEEN (25 mM Tris-HCl, pH 7.4, 1 mM EDTA, 1 mM EGTA, 100 mM NaCl) supplemented with 1 mM phenylmethylsulfonyl fluoride (PMSF). Proteins were resolved on 10% SDS-polyacrylamide gels and transferred to PVDF membranes.

After blocking with 3% bovine serum albumin (BSA), the membrane was incubated with primary antibodies: anti-EAAT-1 (goat polyclonal antibody from Santa Cruz Inc., 1:500) overnight at 4° C.; anti-EAAT-2 (goat polyclonal antibody from Santa Cruz Inc., 1:500) overnight at 4° C.; or anti-actin (mouse monoclonal antibody from Sigma-Aldrich, 1:2000) for 1 hour at room temperature (RT). After washing, the secondary antibody, horseradish peroxidase linked IgG (anti-goat for EAAT-1 and EAAT-2, anti-mouse for actin), was applied at 1:2000 for one hour at RT. The bands were visualized using the enhanced chemiluminescence (ECL) system (Amersham Biosci.).

The results of the Western blot experiments were analyzed with Universal Hood Gel Documentation Systems and Quantity One V4.2.1 software (BIO-RAD).

Cell Count and Statistics:

For each well, pictures were taken for 10 random fields at a magnification of 100×. Cell number (as indicated by the appropriate immunostain) in each of the 10 pictures was counted by an experimenter blind to the condition. Cell numbers in control and treated groups were compared with one-way analysis of variance (ANOVA) using the Instat software program (Graph Pad). Appropriate multiple comparisons tests were performed as indicated in figure legends and the text.

### Example 1

### UA Protects Spinal Cord Neurons from Glutamate-Induced Toxicity

Glutamate is the major physiological agent that mediates cell death after spinal cord injury. To examine the survival of spinal cord neurons in an in vitro model of SCI, mixed cell cultures derived from embryonic 16 (E16) rat spinal cords were treated with a range of concentrations of glutamate. These cells were grown in serum-containing medium (SCM) for 6 days (DIV 6) before being treated with various concentrations of glutamate for 1 hour. The brief exposure to high levels of glutamate mimics its temporary up-regulation after SCI. After glutamate was removed, cells were kept in SCM for another 24 hours before the cultures were fixed and immunostained. The numbers of surviving neurons were monitored with immunohistochemical labeling of MAP-2, a marker for neurons.

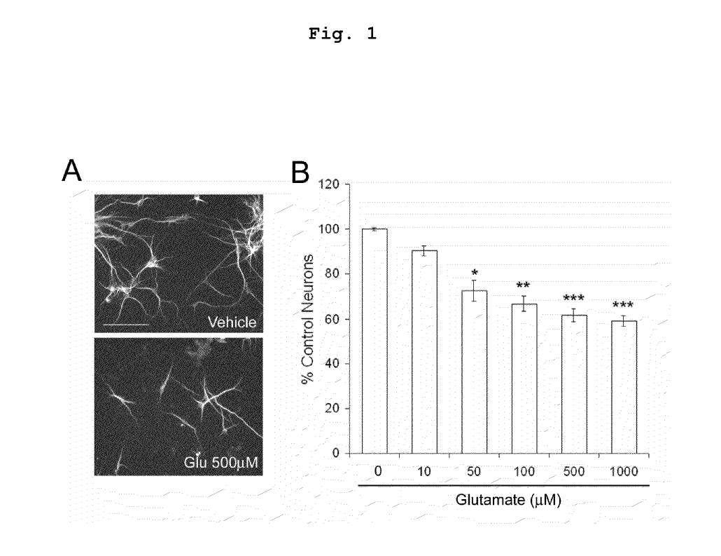

The staining for MAP-2 demonstrated that the surviving neurons bear multiple processes and intact membrane structures (FIG. 1A). As shown in FIG. 1B, glutamate-elicited neuronal cell death increased in a dose-dependent manner. With high concentrations of glutamate (500 μM and 1 mM), approximately 40% of the neurons in these cultures did not survive (FIG. 1B). Results were derived from 3 independent experiments (n=7). *p<0.05, ***p<0.001 by nonparametric ANOVA followed by Dunn's analysis for multiple comparisons comparing glutamate treated groups with control. Scale bar, 50 um.

### Example 2

### UA has No Effect on the Survival of Spinal Cord Neurons

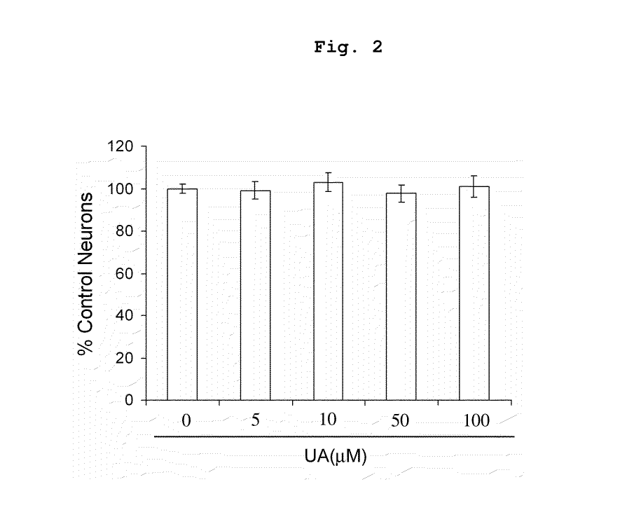

To determine whether UA can be used to prevent glutamate toxicity, initial experiments monitored the effects of UA on spinal cord neuron survival during basal conditions. DIV 6 cultures were treated with different concentrations of UA for 24 hours before being fixed and immunostained, and the numbers of neurons were counted. UA had no effect on the survival of spinal cord neurons (FIG. 2). The results were derived from 2 independent experiments (n=6).

### Example 3

### UA Reduces Neuronal Cell Death after Glutamate Exposure

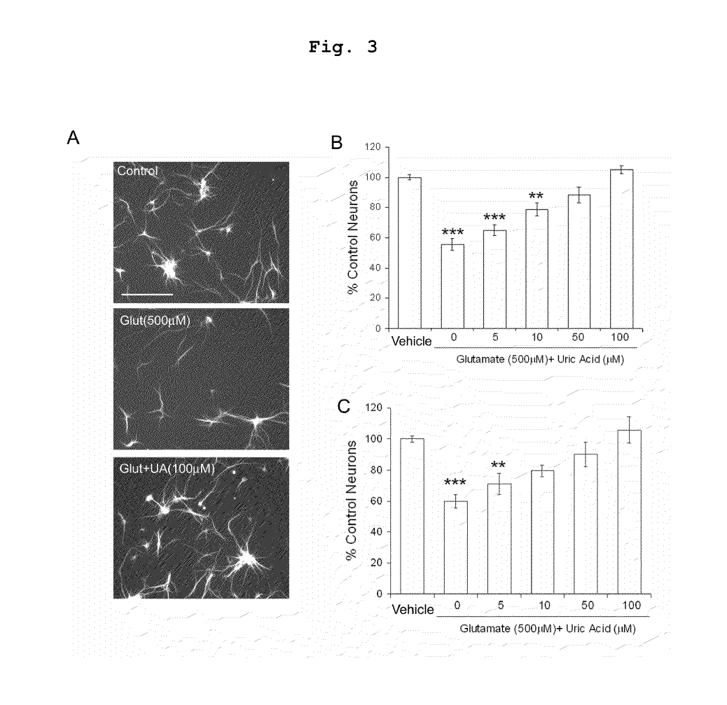

Further experiments were designed to reveal if UA could reduce neuronal cell death after glutamate exposure. Cells were treated with 500 μM glutamate with or without various concentrations of UA. UA was applied with glutamate and the same concentration of UA was added after the removal of glutamate. As shown in FIG. 3, UA blocked glutamate toxicity in a dose-dependent manner. With high concentrations of UA, glutamate-promoted neuronal cell death was abolished (FIG. 3B). The results were derived from 6 independent experiments (n=12).

Most importantly, treatment of UA solely after the termination of glutamate exposure resulted in similar neuroprotection (FIG. 3C). The results were derived from 3 independent experiments (n=9). *p<0.05, **p<0.01, ***p<0.001 by nonparametric ANOVA followed by Dunn's analysis for multiple comparisons using vehicle as control. Scale bar, 50 μm. These results suggest that UA can act to protect neurons after glutamate exposure in culture.

### Example 4

### UA Cannot Protect Spinal Cord Neurons from Glutamate Toxicity in Pure Neuron Cultures

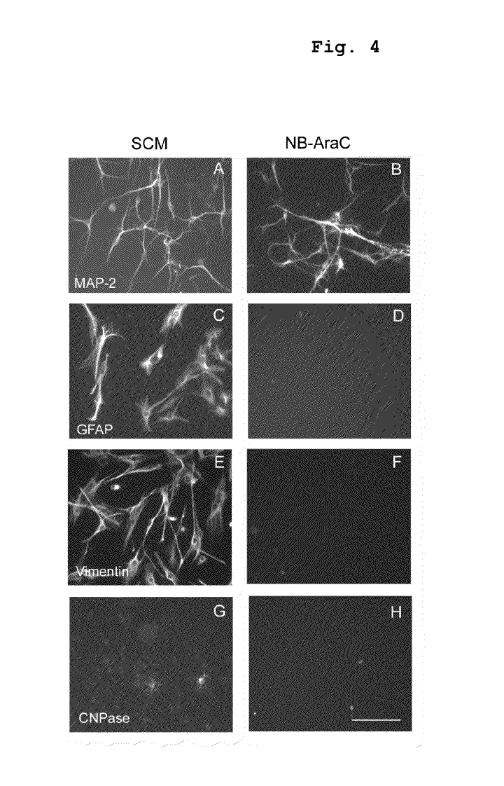

Spinal cord cells grown in SCM are mixed cultures. To identify the cell types present, DIV 6 cultures were immunostained for MAP-2, GFAP, vimentin, CNPase, and CD11. MAP-2 is a marker for neurons, GFAP and vimentin are intermediate filament proteins that are used as markers for mature and immature astrocytes respectively (Yang et al. 1994), CNPase is a marker for oligodendrocytes, and CD11 is a marker for microglia. While MAP-2+ neurons, GFAP+ or vimentin+ astroglia, and CNPase+ oligodendrocytes were identified (FIG. 4A, C, E, G), staining for CD11 was negative (data not shown), indicating the absence of microglia in these cultures.

The presence of astroglia and oligodendrocytes in mixed cultures suggests the possibility that the effects of UA may not be mediated directly by neurons, but by glial cells which indirectly confer neuronal protection. To address this question, pure spinal cord neuron cultures were established. Twenty four hours after plating in SCM, SCM was changed to NB medium, which optimizes neuronal growth. Ara-C (5 μM) was added to the cultures after another 24 hours to eliminate the glial populations. The medium was changed after 3 days, and cells were treated 24 hours later. This procedure eliminated non-neuronal cell types in the cultures with only neurons surviving. Like the neurons grown in SCM, the pure neuron cultures demonstrated that the neurons had extended processes and have intact membrane structures (FIG. 4 B). However, staining for GFAP, vimentin, CNPase (FIG. 4 D, F, H), and CD11 was absent (data not shown), suggesting that these cultures are a pure neuronal population.

### Example 5

### Pure Neuronal Cultures are Much More Sensitive to Glutamate Toxicity than Mixed Cultures

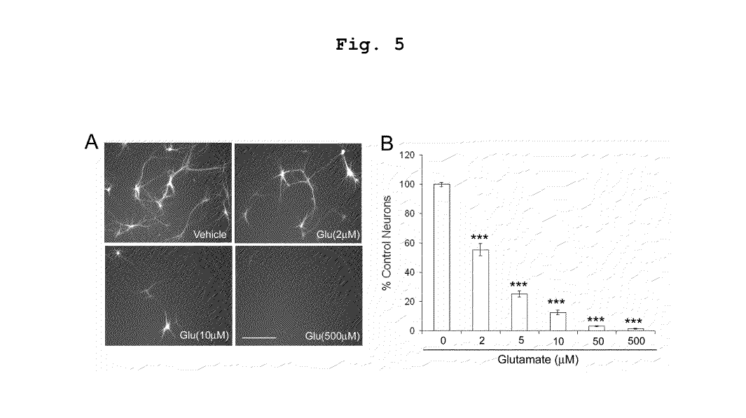

To determine the sensitivity of pure neuronal and mixed cultures to glutamate, pure spinal cord neuron cultures were established, and after 6 days, these cells were treated with glutamate for 1 hour. Cells were fixed after 24 hours and stained with anti-MAP-2 antibody. (A) Control cultures and cells treated with 2, 10, and 500 μM glutamate are illustrated. Treatment with 10 μM glutamate resulted in very significant neuron loss (more than 80%; FIG. 5B), and 500 μM glutamate essentially eliminated all neurons (FIG. 5B), compared to only 40% neuronal loss in the mixed cultures treated with the same concentration of glutamate (500 μM; FIG. 1). Results were derived from 3 independent experiments (n=7). ***p<0.001 by nonparametric ANOVA followed by Dunn's analysis comparing glutamate treated groups with control. Scale bar, 50 μm.

### Example 6

### UA Itself has No Effect on Neuron Survival or Glutamate-Induced Toxicity

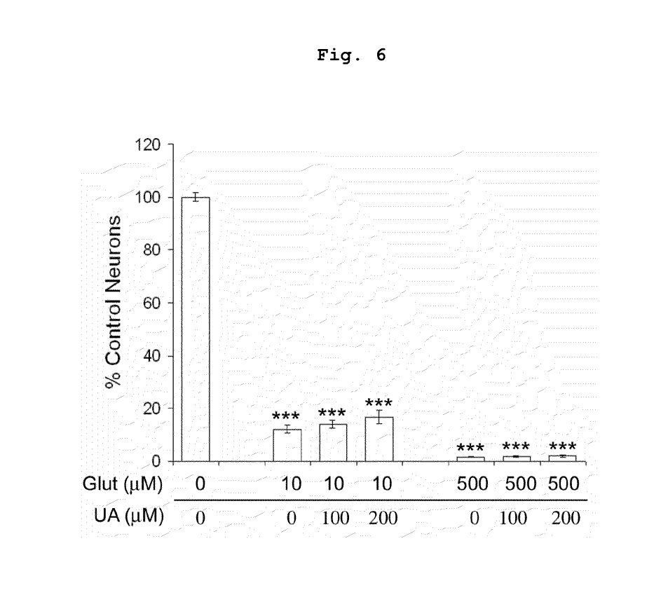

To examine whether UA can directly reduce glutamate toxicity in pure neuronal cultures, cells were treated with or without various concentrations of UA either concurrent with or at the termination of exposure to 10 μM glutamate. More specifically, six days after plating, pure spinal cord neuron cultures were treated with glutamate (10 or 500 μM) for 1 hour. UA (100 or 200 μM) was added with and after glutamate treatments. Cells were fixed after 24 hours and numbers of neurons that survived (MAP-2+) were counted. Results shown in FIG. 6 were derived from 2 independent experiments (n=6). ***p<0.001 by nonparametric ANOVA followed by Dunn's analysis for multiple comparisons comparing treated groups with control. UA itself had no effect on neuron survival, and in addition, it did not show any protection against glutamate toxicity.

### Example 7

### UA Reverses Toxicity Induced by SIN-1

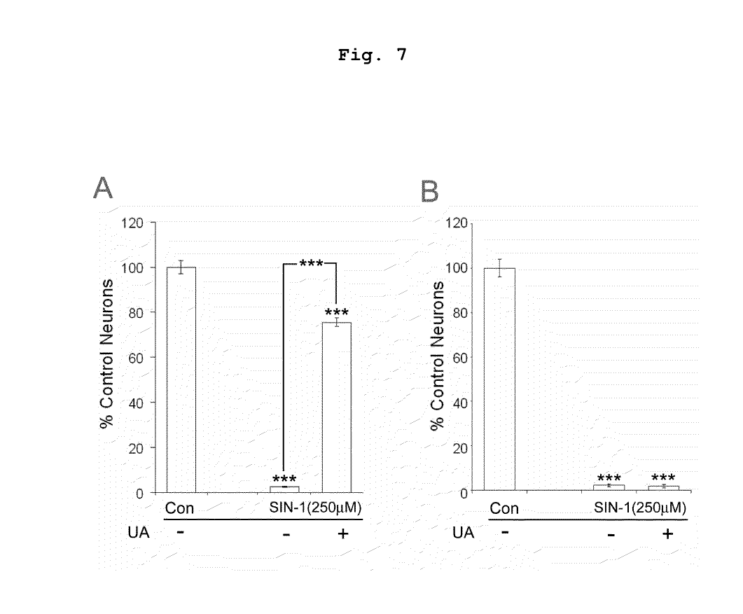

UA is generally considered to be a natural scavenger for peroxynitrite, a reactive oxygen species (ROS) that plays an important role in mediating tissue damage and cell loss in CNS injury and trauma (Keynes and Garthwaite 2004). Thus, the role of UA as a compound against toxicity has been studied rigorously as a direct scavenger for ROS. Neuroprotection elicited by UA has been considered as a result of the reduction of ROS, which directly damage neurons. For example, studies using hippocampal neuronal cultures indicated that the neuroprotective effects of UA involve suppression of oxyradical accumulation (Yu et al. 1998). Moreover, previous findings by Scott and colleagues demonstrated that treatment of mouse spinal cord neuron cultures with UA blocked peroxynitrite toxicity (Scott et al. 2005). Furthermore, pre-treatment and concurrent treatment with UA reduced secondary damage in a mouse model of spinal cord injury (Scott et al. 2005). The ability of UA to dramatically block peroxynitrite toxicity was demonstrated in this study as well (FIG. 7), suggesting that this pathway is regulated by the presence of UA.

As demonstrated in FIG. 7, high concentrations of UA (up to 200 μM) did not alter the neuron loss elicited by 10 μM glutamate. In contrast, toxicity elicited by the peroxynitrite donor, Sin-1 (1-hr treatment at 250 μM), was significantly reversed by the concurrent presence of UA (100 μM) (FIG. 7A). However, when UA was added after Sin-1, it did not elicit a reversal of toxicity (FIG. 7B). Results were derived from 2 independent experiments (n=6). ***p<0.001 by parametric ANOVA followed by Bonferroni Multiple Comparisons Test.

These data suggest that peroxynitrite is probably not the major mediator of glutamate-induced toxicity since 1) UA can protect against Sin-1 toxicity while having no effect on glutamate-induced toxicity in pure neuronal cultures and 2) UA cannot protect neurons from Sin-1-induced toxicity when added after Sin-1 exposure. In addition, these data demonstrate that UA is not likely to affect neurons directly. Non-neuronal cells, most likely astroglia, may mediate the effects of UA to protect neurons from glutamate treatment.

### Example 8

### Astroglia is Involved in Mediating the Effects of UA

One candiate cell type that may mediate the effects of UA is the astroglial population. The roles of astroglia after spinal cord injury have been studied rigorously. Astroglia, especially reactive astrocytes, have been found to form the glia scar, generally considered as a major impediment to axon regeneration (Liuzzi and Lasek 1987; Rudge and Silver 1990).

However, more recent studies indicate that astrocytes may protect tissue and preserve function after SCI (Faulkner et al. 2004; Silver and Miller 2004). Astroglia, including GFAP+ and vimentin+ cells, have been reported to protect neurons from excitotoxic insults in CNS trauma (Diaz et al. 2005; Faulkner et al. 2004). It has also been found that cultured spinal cord astrocytes express glutamate metabotropic receptors. For example, mGluR1 and mGluR2/3 are expressed by a subset of astrocytes derived from rat spinal cords (Silva et al. 1999). As such, the further studies examined whether astroglia contribute to the effects of UA.

To establish pure spinal cord astroglia cultures, cells from P1 rat spinal cord were plated and grown in high serum conditions for 9 days before undergoing sequential shaking procedures to remove microglia and oligodendrocytes. After 3 more days in Ara-C supplemented medium, cells were replated and these spinal cord astroglia cultures consist of GFAP+ and vimentin+ cells, which were also observed in the mixed cultures. There are no neurons or oligodendrocytes present in these cultures (data not shown).

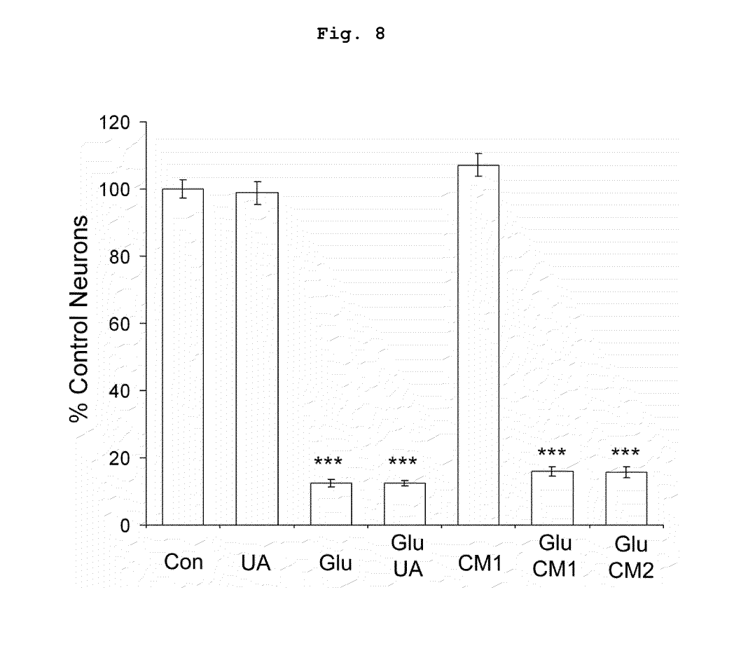

Preliminary experiments were designed to examine whether UA elicits the secretion of soluble factors that contribute to the effects of UA. Astroglia cultures were grown in NB medium for 3 days and treated with UA or Locke's buffer. Conditioned medium (CM) from these cultures were collected 24 hours later. CM from the vehicle-treated group was designated as CM1. CM from UA treated group was CM2. The possible effects of CM1 and CM2 to rescue pure spinal cord neurons from glutamate toxicity were examined. The results derived from 2 independent experiments (n=6) suggest that neither CM1 nor CM2 reduced the damage to neurons elicited by 10 μM glutamate (FIG. 8). Therefore, it is likely that that soluble factors are not likely to be involved in mediating UA actions. ***p<0.001 by parametric ANOVA followed by Bonferroni Multiple Comparisons Test.

### Example 9

### Astroglia is Essential for Mediating the Effects of UA

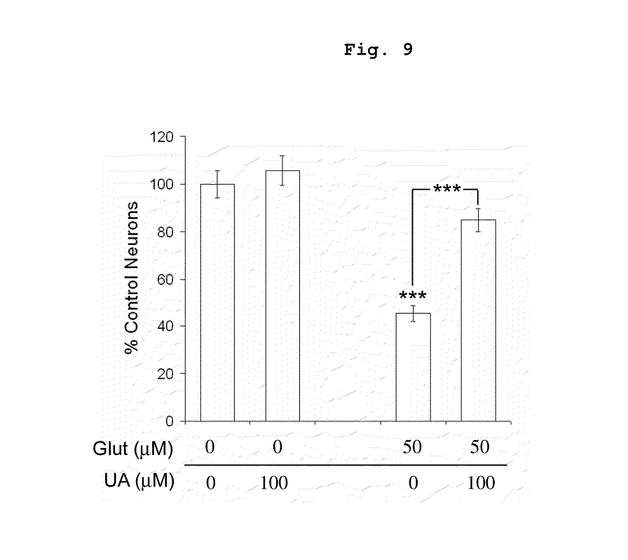

Further studies explored whether direct addition of astroglia to pure neuronal cultures could restore the effects of UA in protection from glutamate toxicity. These cells were grown for 5 days in SCM and then trypsinized and replated onto DIV 5 pure spinal cord neuron cultures. The medium for the combined cultures was changed to SCM. Twenty four hours later, the combined cultures were treated with glutamate (50 μM for 1 hour) followed by UA or vehicle addition. Examination of neuron numbers indicated that 100 μM UA blocked the toxic effects of glutamate (FIG. 9), suggesting that the presence of astroglia is essential for mediating the effects of UA. Results were derived from 2 independent experiments (n=6). ***p<0.001 by parametric ANOVA followed by Bonferroni Multiple Comparisons Test.

### Example 10

### EAAT Expressed by Astroglia May Play an Important Role in Mediating the Neuroprotective Effect of UA

One likely candidate for mediating the effects of UA is the glutamate transporters. These transporters have been shown to protect neurons from glutamate toxicity (Rothstein et al. 1993). Moreover, there are studies demonstrating that EAAT-1 and EAAT-2 are acutely up-regulated after spinal cord injury (Vera-Portocarrero et al. 2002).

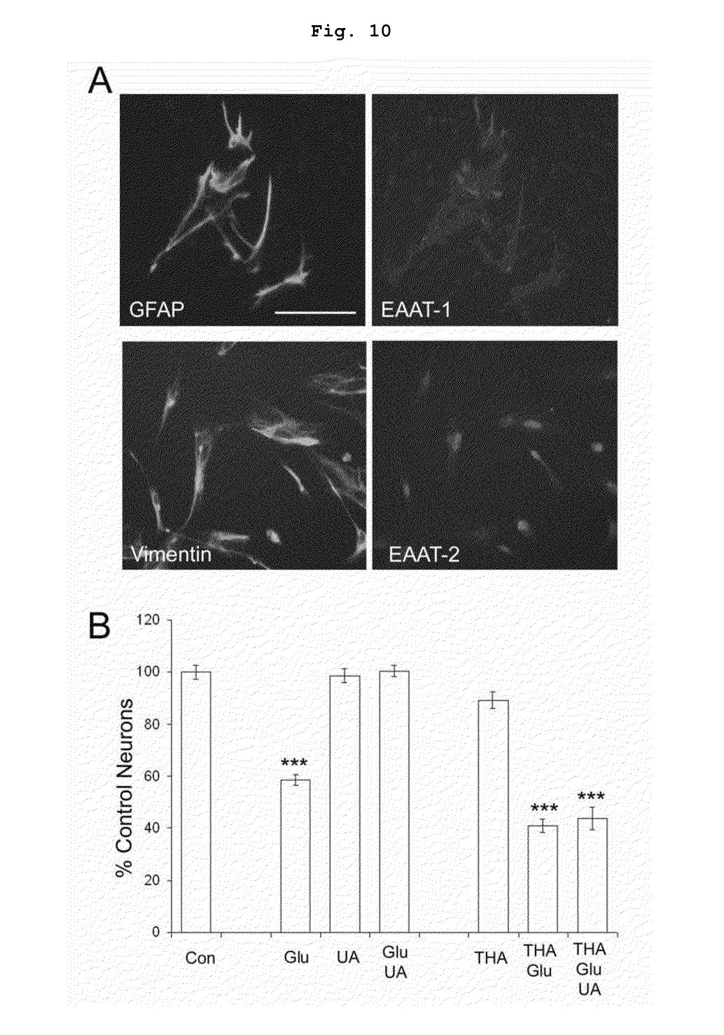

Astroglial cells have been reported to express excitatory amino acid transporters (EAATs), mainly EAAT-1 and EAAT-2. EAATs can remove extracellular glutamate and limit neuronal access to toxicity. Immunostaining studies indicated that EAATs are exclusively expressed by astroglial cells in the instant study. Interestingly, EAAT-1 is co-localized with GFAP+ astroglia and EAAT-2 is expressed by vimentin+ astroglial precursors in the mixed spinal cord cultures (FIG. 10A). Furthermore, blockade of the EAAT activity by inhibitor L-Threohydroxy aspartate (THA, 50 μM added for one hour prior to the addition of glutamate at 50 μM) results in elimination of neuroprotective effect of UA (100 μM) in reducing glutamate toxicity (FIG. 10B), suggesting that EAATs expressed by astroglia may play an important role in mediating the neuroprotective effects of UA.

### Example 11

### The Amount of EAAT-1 Protein is Increased by UA

Additional studies examined whether UA affects protein expression of EAAT-1 and EAAT-2. Mixed spinal cord cultures (DIV 6) were treated with UA (100 μM, 24 hours), glutamate (500 μM, 1 hour treatment, changed back to SCM for 24 hours), or glutamate and UA (UA added after glutamate).

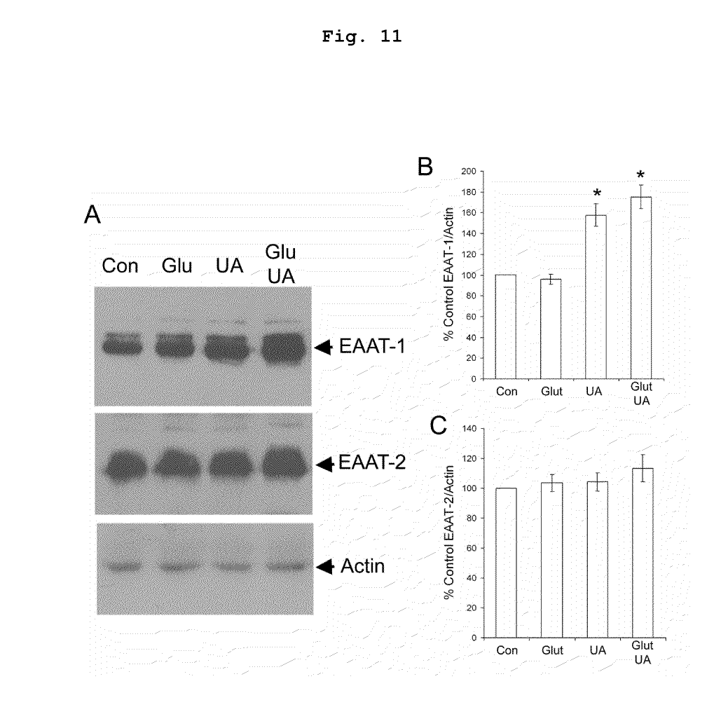

Protein levels of EAAT-1, and EAAT-2 were normalized to actin expression and compared to control (FIG. 11). (A), Western blot of EAAT-1, EAAT-2 and actin. Figure is representative of 5 experiments with similar results.

While EAAT-2 expression was not altered by the treatment (FIG. 11C), EAAT-1 was upregulated by UA (FIG. 11B). Treatment of glutamate prior to UA did not influence UA actions in increasing EAAT-1 protein expression (FIG. 11B). Results were derived from 5 independent experiments. *,p<0.01 by parametric ANOVA followed by Bonferroni Multiple Comparisons Test compared to control group.

These data demonstrated a possible mechanism by which UA acts on astroglia to indirectly protect neurons from glutamate toxicity.

Discussion

The possible roles of UA in protecting neurons were tested against 500 μM glutamate, mimicking the concentration observed in rat spinal cord injury models (McAdoo et al. 1999). In spinal cord cultures grown in SCM, the applicants demonstrated that UA, a ubiquitous anti-toxicant, protected spinal cord neurons against glutamate toxicity. A concentration of 100 μM UA reversed the cell loss elicited by treatment with 5001.1M glutamate (FIG. 3). It should be noted that cultures grown in SCM are a mixed population. Together with neurons, oligodendrocytes and astroglial cells were present in these cultures (FIG. 4). Astroglial cells were identified with GFAP and vimentin, intermediate filament proteins that are used as markers for mature and immature astrocytes (Yang et al. 1994). Further double-labeling studies indicated that GFAP and vimentin immunostaining were not observed in the same cells in the majority of cells examined (data not shown), demonstrating two cell populations in the mixed spinal cord culture. To evaluate whether oligodendrocytes and astroglia are involved in UA actions in neuronal protection, pure spinal cord neuron cultures were developed, and the neuroprotective effect of UA was evaluated. Interestingly, UA did not protect spinal cord neurons against glutamate toxicity in these cultures (FIG. 6). The lack of UA action on pure neurons suggests that glial cells are essential for reducing the damage to spinal cord neurons.

It should also be noted that a recent study of UA-peroxynitrite binding kinetics revealed that at normal human levels, carbon dioxide binds with peroxynitrite 920 times faster than UA, suggesting that it is unlikely that UA plays a major role in reducing peroxynitrite toxicity in vivo (Squadrito et al. 2000).

The instant disclosure provides a possible alternative mechanism for UA actions and suggest an essential glial involvement in the effects of UA. In the SCM spinal cord cultures, neurons were very resistant to the toxic effects of glutamate. There was only about 40% neuron loss after one-hour treatment with 500 μM glutamate (FIG. 1) compared to almost total neuron elimination in the pure cultures (FIG. 5). This is probably due to the presence of astroglial cells and their likely roles to protect neurons in these cultures. In fact, previous reports have demonstrated that astroglial cells can reduce damage to neurons through distinct mechanisms. For example, early studies indicated that glutamate toxicity was much more potent in cortical neurons grown in an astrocyte-poor culture than those grown in an astrocyte-rich culture (Rosenberg et al. 1992). Astroglial cells have also been shown to secrete neuroprotective factors such as transforming growth factor beta (TGF-β; (Dhandapani and Brann 2003) and brain-derived neurotrophic factor (BDNF; (Dougherty et al. 2000; Wu et al. 2004). Interestingly, adding conditioned media from UA-treated astroglial cultures did not rescue the neuroprotective effects in pure neuronal cultures (FIG. 8), suggesting that in order for UA to act as a neuroprotectant, 1) astroglia must be physically present to rescue neurons from glutamate-induced death or 2) there must be some cross-talk between neurons and astroglia, possibly by secreted factors. In fact, the direct presence of astroglial cells significantly reduced glutamate damage (FIG. 2 and FIG. 5). Furthermore, the seeding of astroglial cells into pure spinal cord neuron culture re-instated the ability of UA to protect neurons from glutamate toxicity (FIG. 9), suggesting that UA not only mediates its neuroprotective effects indirectly by acting on astroglia, but also requires the direct presence of astroglia in the cultures.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

All patent and non-patent publications cited in this specification are indicative of the level of skill of those skilled in the art to which this invention pertains. All these publications and patent applications are herein incorporated by reference to the same extent as if each individual publication or patent application was specifically and individually indicated to be incorporated herein by reference.

1. A kit for identifying compounds reducing excitotoxicity in neurons, comprising:
a) a plurality of samples of glial cells;
b) a means for determining the amount of EAAT-1 protein present on a cell surface; and
c) a set of instructions.

2. A kit for identifying compounds reducing excitotoxicity in neurons, comprising:
a) a plurality of samples of glial cells;
b) a means for determining the amount of EAAT-1 protein present in a cell; and
c) a set of instructions.

3. The kit of claim 1, further comprising uric acid.

4. The kit of claim 1, wherein said glial cells are astroglial cells.

5. The kit of claim 1, wherein said means comprise anti-EAAT-1 antibodies.

6. The kit of claim 5, wherein said glial cells are astroglial cells.

7. The kit of claim 2, wherein said glial cells are astroglial cells.

8. The kit of claim 2, wherein said means comprise anti-EAAT-1 antibodies.

9. The kit of claim 3, wherein said means comprise anti-EAAT-1 antibodies.

10. The kit of claim 4, wherein said means comprise anti-EAAT-1 antibodies.

11. The kit of 2, further comprising uric acid.

12. The kit of claim 11, wherein said means comprise anti-EAAT-1 antibodies.

13. The kit of claim 7, wherein said means comprise anti-EAAT-1 antibodies.

14. The kit of claim 13, wherein said glial cells are astroglial cells.


SEQUENCE LISTING (3 sequences)

SEQ ID NO: 1 - protein, 542 aa, Homo sapiens
MTKSNGEEPKMGGRMERFQQGVRKRTLLAKKKVQNITKEDVKSYLFRNAFVLLTVTAVIVGTILGFTLRPYRMSYREVKYFSFPGELLMRMLQMLVLPLIISSLVTGMAALDSKASGKMGMRAVVYYMTTTIIAVVIGIIIVIIIHPGKGTKENMHREGKIVRVTAADAFLDLIRNMFPPNLVEACFKQFKTNYEKRSFKVPIQANETLVGAVINNVSEAMETLTRITEELVPVPGSVNGVNALGLVVFSMCFGFVIGNMKEQGQALREFFDSLNEAIMRLVAVIMWYAPVGILFLIAGKIVEMEDMGVIGGQLAMYTVTVIVGLLIHAVIVLPLLYFLVTRKNPWVFIGGLLQALITALGTSSSSATLPITFKCLEENNGVDKRVTRFVLPVGATINMDGTALYEALAAIFIAQVNNFELNFGQIITISITATAASIGAAGIPQAGLVTMVIVLTSVGLPTDDITLIIAVDWFLDRLRTTTNVLGDSLGAGIVEHLSRHELKNRDVEMGNSVIEENEMKKPYQLIAQDNETEKPIDSETKM

SEQ ID NO: 2 - protein, 543 aa, Mus musculus
MTKSNGEEPRMGGRMERLQQGVRKRTLLAKKKVQSLTKEDVKSYLFRNAFVLLTVTAVIVGTILGFALRPYKMSYREVKYFSFPGELLMRMLQMLVLPLIISSLVTGMAALDSKASGKMGMRAVVYYMTTTIIAVVIGIIIVIIIHPGKGTKENMYREGKIVQVTAADAFLDLIRNMFPPNLVEACFKQFKTSYEKRSFKVPIQSNETLLGAVINNVSEAMETLTRIREEMVPVPGSVNGVNALGLVVFSMCFGFVIGNMKEQGQALREFFDSLNEAIMRLVAVIMWYAPLGILFLIAGKIVEMEDMGVIGGQLAMYTVTVIVGLLIHAVIVLPLLYFLVTRKNPWVFIGGLLQALITALGTSSSSATLPITFKCLEENNGVDKRITRFVLPVGATINMDGTALYEALAAIFIAQVNNFDLNFGQIITISITATAASIGAAGIPQAGLVTMVIVLTSVGLPTDDITLIIAVDWFLDRLRTTTNVLGDSLGAGIVEHLSRHELKNRDVEMGNSVIEENEMKKPYQLIAQDNEPEKPVADSETKM

SEQ ID NO: 3 - protein, 543 aa, Rattus norvegicus
MTKSNGEEPRMGSRMERFQQGVRKRTLLAKKKVQNITKEDVKSYLFRNAFVLLTVSAVIVGTILGFALRPYKMSYREVKYFSFPGELLMRMLQMLVLPLIISSLVTGMAALDSKASGKMGMRAVVYYMTTTIIAVVIGIIIVIIIHPGKGTKENMYREGKIVQVTAADAFLDLIRNMFPPNLVEACFKQFKTSYEKRSFKVPIQANETLLGAVINNVSEAMETLTRIREEMVPVPGSVNGVNALGLVVFSMCFGFVIGNMKEQGQALREFFDSLNEAIMRLVAVIMWYAPLGILFLIAGKILEMEDMGVIGGQLAMYTVTVIVGLLIHAVIVLPLLYFLVTRKNPWVFIGGLLQALITALGTSSSSATLPITFKCLEENNGVDKRITRFVLPVGATINMDGTALYEALAAIFIAQVNNFDLNFGQIITISITATAASIGAAGIPQAGLVTMVIVLTSVGLPTDDITLIIAVDWFLDRLRTTTNVLGDSLGAGIVEHLSRHELKNRDVEMGNSVIEENEMKKPYQLIAQDNEPEKPVADSETKM